(19) United States
(12) Patent Application Publication
(10) Pub. No.: US 2026/0144735 A1
(43) Pub. Date: May 28, 2026

(54) SOLID COSMETIC COMPOSITION CONTAINING LARGE AMOUNT OF HYALURONIC ACID

(71) Applicant: COSMAX, INC, Gyeonggi-do (KR)

(72) Inventors: Na-Seul JUNG, Gyeonggi-do (KR); Hye-Sun CHOI, Gyeonggi-do (KR); Younghwa WOO, Gyeonggi-do (KR); Myeong Sam PARK, Seoul (KR)

(21) Appl. No.: 19/122,795

(22) PCT Filed: Oct. 30, 2023
(86) PCT No.: PCT/KR2023/017012

§ 371 Date: Apr. 21, 2025

(30) Foreign Application Priority Data
Oct. 31, 2022 (KR) 10-2022-0142696

(51) Int. Cl. A61K 8/73; A61K 8/04; A61K 8/92; A61Q 1/06; A61Q 19/00

(52) U.S. Cl. CPC A61K 8/735; A61K 8/04; A61K 8/92; A61Q 1/06; A61Q 19/007; A61K 2800/43

(57) ABSTRACT

The present invention relates to a solid cosmetic composition containing a large amount of hyaluronic acid. The solid cosmetic composition according to the present invention has an excellent moisturizing effect. The present invention also provides a solid cosmetic composition that contains a large amount of hyaluronic acid and also has high stability in oil-dispersion formulations.

### TECHNICAL FIELD

The present disclosure relates to a cosmetic composition containing hyaluronic acid and, specifically, to a solid cosmetic composition in an oil dispersion formulation containing a large amount of hyaluronic acid.

### BACKGROUND ART

Hyaluronic acid is a mucopolysaccharide found in living organisms, especially in subcutaneous tissue, and has been widely used as a raw material for cosmetics in the form of hyaluronic acid or its salt due to excellent moisturizing functions. Hyaluronic acid is essential for forming collagen matrix and elastic fibers and maintaining skin moisture. Hyaluronic acid plays a key role in offering elasticity and density to the skin since hyaluronic acid forms a large molecular complex that provides a type of support structure by combining with collagen, fibronectin, and proteoglycan in the skin. Particularly, low-molecular-weight hyaluronic acid molecules have good skin penetration and high water-holding capacity of binding with water, thereby ensuring increased hydration and skin elasticity.

Conventional hyaluronic acid is a water-soluble raw material and thus has been applied to the aqueous phase of water dispersion formulations or emulsion formulations. However, conventional hyaluronic acid had limitations in its application to cosmetics in an oil dispersion formulation, such as wax-based stick-type cosmetics.

To overcome these limitations, there is a need for the development of solid cosmetic compositions obtained by applying a high content of hyaluronic acid to a cosmetic composition in an oil dispersion formulation.

### DISCLOSURE

### Technical Problem

The present disclosure has been made to solve the above-described problems, and an aspect of the present disclosure is to provide a solid cosmetic composition in an oil dispersion formulation containing a large amount of hyaluronic acid.

Another aspect of the present disclosure is to provide a cosmetic composition in an oil dispersion formulation exhibiting excellent moisturizing effects and high stability of the oil dispersion formulation by maximizing the content of hydronic acid.

### Technical Solution

In accordance with an aspect of the present disclosure, there is provided a solid cosmetic composition containing hyaluronic acid and wax

The cosmetic composition may be an oil dispersion formulation, and the hyaluronic acid may be contained in a content of 1 to 30 wt % relative to the total weight of the composition.

The hyaluronic acid may be at least one selected from the group consisting of sodium hyaluronic acid, hydroxypropyltrimonium hyaluronic acid, sodium acetylated hyaluronic acid, potassium hyaluronic acid, and hydrolyzed hyaluronic acid.

The wax may be at least one selected from the group consisting of beeswax, castor seed oil, hydrogenated castor oil, candelilla wax, carnauba wax, rice bran wax, jojoba wax, montan cera, shellac wax, beeswax, lanolin, ozokerite, ceresin, paraffin, and microcrystalline wax, and the wax may be contained in a content of 5 to 50 wt % relative to the total weight of the composition.

The solid cosmetic composition may further contain at least one selected from the group consisting of an emollient, a surfactant, a preservative, and a pigment.

The emollient may be at least one selected from the group consisting of diisostearyl malate, dicetearyl dimer dilinoleate, hexyl laurate, squalane, pentaerythrityl tetraisostearate, octyldodecyl stearoyl stearate, dimethicone, and phenyl trimethicone, and may be contained in a content of 5 to 40 wt % relative to the total weight of the composition.

The surfactant may be at least one selected from the group consisting of glycerin fatty acid ether, propylene glycol fatty acid ester, sorbitan fatty acid ester, polyglyceryl-10 stearate, and cetearyl glucoside, and may be contained in a content of 0.05 to 10 wt % relative to the total weight of the composition.

### Advantageous Effects

The cosmetic composition according to the present disclosure exhibits excellent moisturizing effects.

Furthermore, the cosmetic composition exhibits outstanding oil dispersibility and formulation stability.

Furthermore, the cosmetic composition provides coloring effects and color retention.

### DESCRIPTION OF DRAWINGS

FIG. 1 shows images depicting color retention test results according to an exemplary embodiment of the present disclosure.

FIG. 2 shows graphs (a) and images (b) depicting moisturizing test results according to an exemplary embodiment of the present disclosure.

FIG. 3 shows graphs depicting relative water content test results according to an exemplary embodiment of the present disclosure.

### BEST MODE FOR INVENTION

Hereinafter, the present disclosure will be described in detail with reference to exemplary embodiments. However, these exemplary embodiments are for illustrative purposes only, and the scope of the present disclosure is not intended to be limited thereto.

An aspect of the present disclosure provides an oil-dispersed solid cosmetic composition containing hyaluronic acid and wax.

The solid cosmetic composition may further contain an emollient, a surfactant, a preservative, a pigment, and/or other additives.

In an embodiment, the solid cosmetic composition may be used for eye makeups, lipsticks, blushers, sun-sticks, and concealers.

### A. Hyaluronic Acid

The solid cosmetic composition of the present disclosure contains hyaluronic acid. The hyaluronic acid may be at least one selected from the group consisting of sodium hyaluronic acid, hydroxypropyltrimonium hyaluronic acid, sodium acetylated hyaluronic acid, potassium hyaluronic acid, and hydrolyzed hyaluronic acid, and may be preferably potassium hyaluronic acid, which is a low-molecular-weight hyaluronic acid.

In the present disclosure, hyaluronic acid may be used in the form of a microsphere that is trapped in a wax matrix. This type of hyaluronic acid has improved dispersibility in the oil phase, enabling its formulation to be stable even though hyaluronic acid, a water-soluble raw material, is contained in large amounts in the oil phase.

In the present disclosure, the hyaluronic acid may be contained in a content of 1 to 30 wt % relative to the total weight of the cosmetic composition. In a preferable embodiment, the content of the hyaluronic acid of the present disclosure is preferably 1 wt % or more, 2 wt % or more, 3 wt % or more, 5 wt % or more, 8 wt % or more, 10 wt % or more, or 12 wt % or more, and preferably 30 wt % or less, 25 wt % or less, 20 wt % or less, 15 wt % or less, 10 wt % or less, or 5 wt % or less. Therefore, hyaluronic acid can achieve formulation stability and dispersibility.

### B. Wax

The solid cosmetic composition may contain a wax. The wax may include any wax commonly known in the art, and may be, for example, at least one selected from the group consisting of a plant wax, an animal wax, a mineral wax, a petroleum wax, a synthetic wax, and a mixture thereof.

Specifically, the wax may be at least one selected from the group consisting of beeswax, castor seed oil, hydrogenated castor oil, candelilla wax, carnauba wax, rice bran wax, jojoba wax, montan cera, shellac wax, beeswax, lanolin, ozokerite, ceresin, paraffin, and microcrystalline wax.

In the composition of the present disclosure, the wax may be contained in a content of 5 to 50 wt %. In a preferable embodiment, the content of the wax of the present disclosure is preferably 5 wt % or more, 10 wt % or more, 15 wt % or more, 20 wt % or more, or 25 wt % or more, and preferably 50 wt % or less, 45 wt % or less, 40 wt % or less, 35 wt % or less, or 30 wt % or less. In the present disclosure, less than 5 wt % of the wax makes it difficult to formulate the composition into a solid, such as a stick. In the present disclosure, more than 50 wt % of the wax causes the composition to become too hard, resulting in a stiff and rigid texture with decreased spreadability.

### C. Emollient

The solid cosmetic composition may contain an emollient. In the present disclosure, the emollient prevents moisture evaporation by forming an oil film on the skin.

The emollient may include any emollient commonly known in the art, and may be, for example, at least one selected from the group consisting of diisostearyl malate, dicetearyl dimer dilinoleate, hexyl laurate, squalane, pentaerythrityl tetraisostearate, octyldodecyl stearoyl stearate, dimethicone, and phenyl trimethicone.

In the composition of the present disclosure, the emollient may be contained in a content of 5 to 40 wt %. In a preferable embodiment, the content of the emollient of the present disclosure is preferably 5 wt % or more, 8 wt % or more, 10 wt % or more, 5 wt % or more, 12 wt % or more, 15 wt % or more, or 20 wt % or more, and preferably 40 wt % or less, 37 wt % or less, 35 wt % or less, 32 wt % or less, 30 wt % or less, or 25 wt % or less. Therefore, the emollient can achieve the uniform mixing of components, offer rigidity and strength suitable for molding into and maintaining a solid formulation, especially, a stick type, and improve the feel in use of the cosmetic composition.

### D. Surfactant

The solid cosmetic composition may contain a surfactant. In the present disclosure, the surfactant improves the dispersibility to affect the stability of a formulation.

The surfactant may include any surfactant known in the art, and may be for example at least one selected from the group consisting of glycerin fatty acid ether, propylene glycol fatty acid ester, sorbitan fatty acid ester, polyglyceryl-10 stearate, and cetearyl glucoside.

In the composition of the present disclosure, the surfactant may be contained in a content of 0.05 to 10 wt %. In a preferable embodiment, the content of the surfactant of the present disclosure is preferably 0.05 wt % or more, 0.1 wt % or more, 0.5 wt % or more, 1 wt % or more, 2 wt % or more, 3 wt % or more, or 5 wt % or more, and preferably 10 wt % or less, 9 wt % or less, 8 wt % or less, 6 wt % or less, 5 wt % or less, or 3 wt % or less. Therefore, the surfactant can improve the dispersibility and ensure the formulation stability.

### E. Preservative

The solid cosmetic composition may contain a preservative. The preservative may include any preservative known in the art, and may be for example at least one selected from the group consisting of alcohol, isopropanol, propanediol, 1,2-hexanediol, chlorobutanol, benzyl alcohol, ethyl hexyl glycerin, phenoxyethanol, hydroxyacetophenone, pentylene glycol, and glyceryl caprylate.

In the composition of the present disclosure, the preservative may be contained in a content of 0.03 to 1.5 wt %.

### F. Pigment

The solid cosmetic composition may contain a pigment. The pigment may include any pigment known in the art, and may be for example an inorganic pigment, an organic pigment, or a combination thereof.

In the composition of the present disclosure, the pigment may be contained in a content of 0.5 to 30 wt %.

### G. Other Additives

The cosmetic composition may further contain an additive commonly used in the art, to which the present disclosure pertains, according to use. Preferably, the additive may include at least one selected from the group consisting of a thickener, a preservative, an antioxidant, a flavoring agent, a sequestrant, and a UV filter, but is not limited thereto.

In the composition of the present disclosure, the above-described additive may be contained in a content of 5 to 20 wt %.

### MODE FOR INVENTION

Hereinafter, preferable examples are set forth for better understanding of the present disclosure. However, the following exemplary embodiments are merely for illustrating the present disclosure, and are not intended to limit the scope of the present disclosure.

### <Preparation of Cosmetic Composition>

Cosmetic compositions with varying hyaluronic acid contents were prepared according to the component proportions (wt %) in Table 1.

TABLE 1

| | Comparative | | |
|---|---|---|---|
| Component (wt %) | Example | Example 1 | Example 2 |
| Glyceryl caprylate | 0.05 | 0.05 | 0.05 |
| Microcrystalline wax | 7.4 | 7.4 | 7.4 |
| Bis-diglyceryl polyacyladipate-2 | 7.5 | 7.5 | 7.5 |
| Cetyl ethylhexanoate | 20.0 | 20.0 | 20.0 |
| Sucrose tetrastearate triacetate | 1.0 | 1.0 | 1.0 |
| Squalane | 5.0 | 5.0 | 5.0 |
| Carnauba wax | 0.045 | 0.045 | 0.045 |
| Caprylyl glycol | 0.05 | 0.05 | 0.05 |
| Candelilla wax | 4.255 | 4.255 | 4.255 |
| Tocopherol | 0.00005 | 0.00005 | 0.00005 |
| Paraffin | 3.60 | 3.60 | 3.60 |
| Polyglyceryl-2 triisostearate | 2.505 | 2.505 | 2.505 |
| Castor oil | To 100 | To 100 | To 100 |
| Phytosteryl/Isostearyl/Cetyl/ | 5.0 | 5.0 | 5.0 |
| Stearyl/Behenyl Dimer Dilinoleate | | | |
| Hydrogenated castor oil | 0.0 | 0.668 | 2.0 |
| Hydrogenated vegetable oil | 2.0 | 2.0 | 2.0 |
| Hydrogenated polyisobutene | 14.99995 | 14.99995 | 14.99995 |
| Synthetic wax | 1.2 | 1.2 | 1.2 |
| Red iron oxide (CI 77491) | 0.005 | 0.005 | 0.005 |
| Hyaluronic acid | 0.0 | 1.0 | 3.0 |
| Total | 100.0 | 100.0 | 100.0 |

Specifically, the components were weighed according to the component proportions in Table 1, and then the waxes, emollients, surfactant, and preservative were mixed using a disper at 80 to 100° C. for about 20 minutes. Subsequently, a pigment and other additives were introduced thereinto, and then dispersed using a disper at 80 to 100° C. for about 10 minutes. Hyaluronic acid, hydrogenated castor oil, and caster seed oil were added, and then dispersed using a disper at 80 to 100° C. for about 5 minutes. Thereafter, the resultant mixture was cooled at 4° C. to manufacture a lipstick.

### <Stability Test>

The cosmetic compositions of the examples and comparative example were subjected to a stability test.

The stability was evaluated by observing formulations under the respective temperature conditions in Table 2 below, and the results are shown.
⊚: very stable, ◯: stable, Δ: unstable, x: phase separation

TABLE 2

| | Comparative | | |
|---|---|---|---|
| Temperature | Example | Example 1 | Example 2 |
| Heated at 80° C. | ⊚ | ⊚ | ⊚ |
| Stored in constant temperature | ⊚ | ⊚ | ⊚ |
| chamber at 25° C. for 3 months | | | |
| Stored in constant temperature | ⊚ | ⊚ | ⊚ |
| chamber at 40° C. for 7 days | | | |
| Stored in three cycles at −10° C., | ⊚ | ⊚ | ⊚ |
| 25° C., and 45° C., with a | | | |
| period of 8 hours for each | | | |
| cycle | | | |

As shown in Table 2, all the examples and the comparative example showed very stable formulations under all the temperature conditions.

### <Color Retention Test>

The cosmetic compositions of the examples and comparative example were subjected to a color retention test.

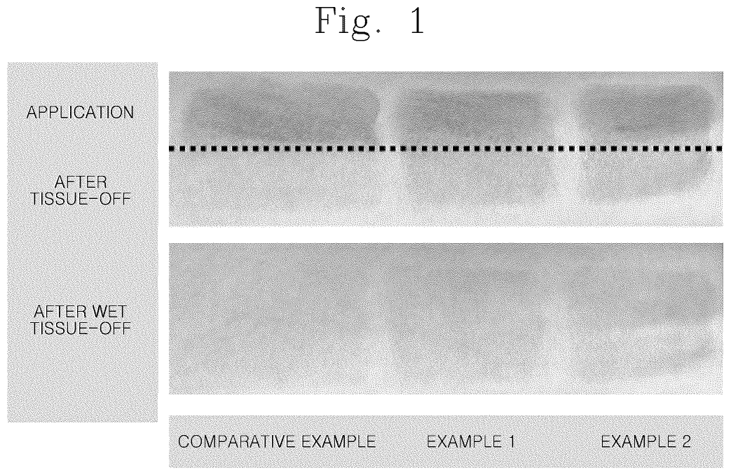

The color retention test was conducted by applying each of the cosmetic compositions prepared from the examples and the comparative example to the inner side of the forearm, wiping off with tissue or wet tissue, and then visually checking for the presence of color retention. FIG. 1 shows the test results.

As shown in FIG. 1, Examples 1 and 2 showed color retaining even after wiped off with tissue and wet tissue, but the comparative example showed almost no color remaining.

It could be confirmed from FIG. 1 that the solid cosmetic composition of the present disclosure had coloring effects and improved color retention.

### <Feel-In-Use Test>

The cosmetic compositions of the examples and comparative example were subjected to a feel-in-use test.

The feel-in-use test was conducted on 20 adults aged 25 to 45, who were asked to use the lipsticks, manufactured in the examples and the comparative example, for 7 days and then to evaluate the feel in use. The color distribution, spreadability, and skin coloring degree was evaluated and recorded based on the following criteria, and the evaluation levels with the highest frequency, among 20 participants, are shown in Table 3 below.

### Evaluation Criteria

⊚: very good, ◯: good, Δ: bad, x: very bad

TABLE 3

| | Comparative | | |
|---|---|---|---|
| | Example | Example 1 | Example 2 |
| Color distribution | ◯ | ◯ | ◯ |
| Spreadabilty | ⊚ | ◯ | ◯ |
| Skin coloring degree | Δ | ◯ | ◯ |

As shown in Table 3, Examples 1 and 2 were evaluated as good in all the items, but the comparative example not containing hyaluronic acid was evaluated as having poor skin coloring degree.

### <Moisturizing Test>

The cosmetic compositions of the examples and comparative example were subjected to a moisturizing test.

The moisturizing test was conducted by comparing the skin moisture level after the application of cosmetic compositions prepared from the examples and the comparative example to the skin, and the skin moisture level before the application. Specifically, after the test site was cleansed, the participants were placed in a constant temperature and humidity room (22±2° C., 40˜60% RH) for 10 minutes, and then the skin moisture level of the test site was measured. Thereafter, the cosmetic composition was applied to the test site, and the skin moisture level was measured shortly after the application. The Epsilon device (BiOX, England) was used as a measurement device, and the measurement was repeated three times. The average and change rate of the measurement values were calculated, and the results are shown in Table 2.

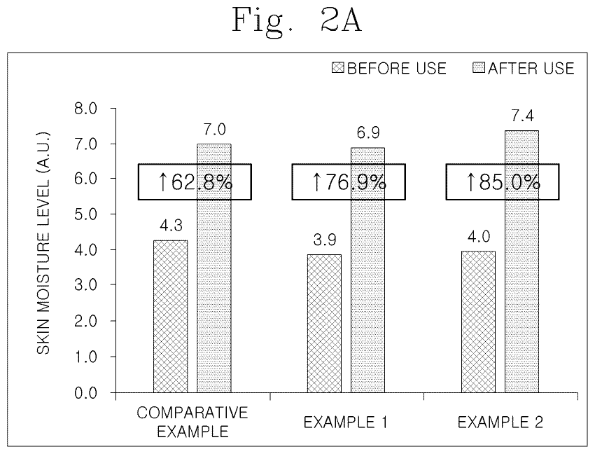

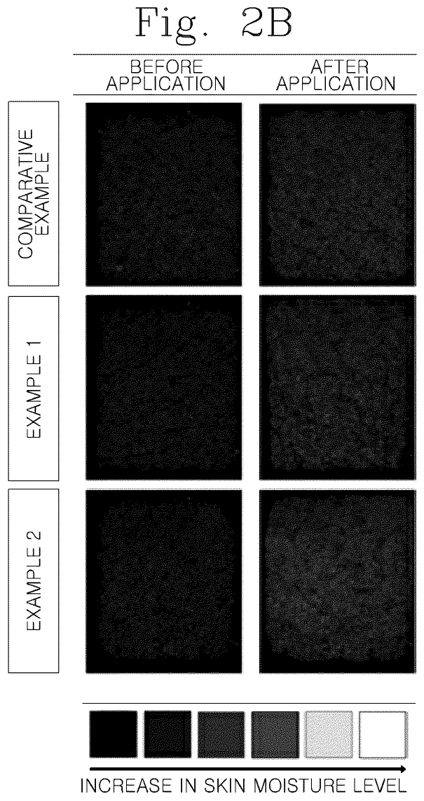

As shown in FIG. 2, the higher the content of hyaluronic acid, the higher the increase rate of the skin moisture level after use. Particularly, Example 2 containing 3 wt % of hyaluronic acid showed an 85% increase in skin moisture level after use, indicating excellent moisturizing effects.

### <Relative Water Content Test>

The cosmetic compositions of the example and comparative example were subjected to relative water content test.

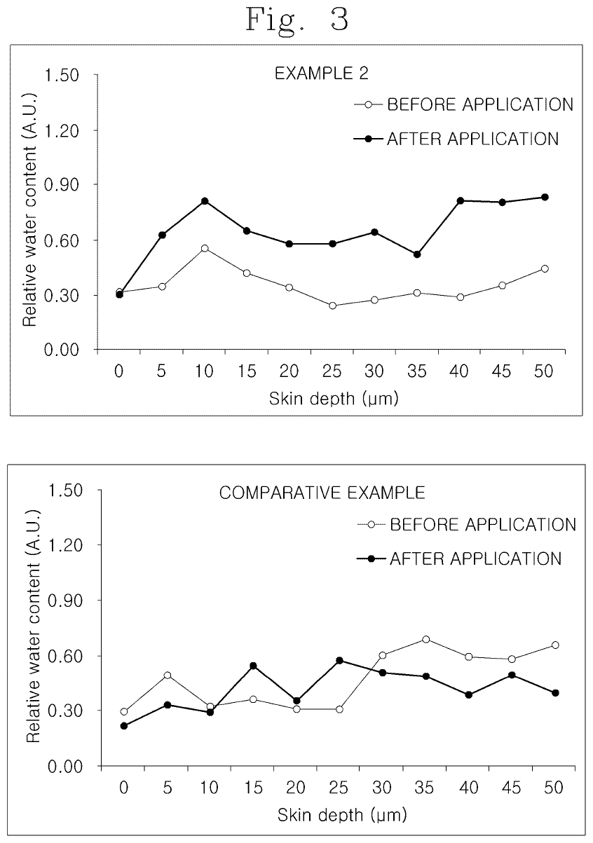

The relative water content test was conducted by applying each of the cosmetic compositions prepared from Example 2 and the comparative example to the inner side of the forearm and then measuring the relative water contents depending on the depth before the application and 20 minutes after the application. A Raman spectroscopy from HORIBA (France) was used as a measurement device. The average relative water content depending on the depth measured on three subjects was used to calculate the relative water content depending on the depth from the skin surface to a depth of 50 μm. The results are shown in FIG. 3.

As shown in FIG. 3, the application of Example 2 containing 3 wt % of hyaluronic acid showed a high relative water content to a depth of 50 μm. That is, it can be seen from the relative water content test that the cosmetic composition of the present disclosure increases the relative water content deep into the skin, beyond the stratum corneum.

FIGS. 2 and 3 indicate that the cosmetic composition of the present disclosure exhibits excellent moisturizing effects on the skin surface as well as deep into the skin.

### INDUSTRIAL APPLICABILITY

The present disclosure is applicable in cosmetics.

What is claimed is:

1. An oil-dispersed solid cosmetic composition comprising hyaluronic acid and wax,
wherein the hyaluronic acid is contained in a content of 1 to 30 wt % relative to the total weight of the composition.

2. The solid cosmetic composition of claim 1, wherein the hyaluronic acid is at least one selected from the group consisting of sodium hyaluronic acid, hydroxypropyltrimonium hyaluronic acid, sodium acetylated hyaluronic acid, potassium hyaluronic acid, and hydrolyzed hyaluronic acid.

3. The solid cosmetic composition of claim 1, wherein the wax is at least one selected from the group consisting of beeswax, castor seed oil, hydrogenated castor oil, candelilla wax, carnauba wax, rice bran wax, jojoba wax, montan cera, shellac wax, beeswax, lanolin, ozokerite, ceresin, paraffin, and microcrystalline wax.

4. The solid cosmetic composition of claim 1, wherein the wax is contained in a content of 5 to 50 wt % relative to the total weight of the composition.

5. The solid cosmetic composition of claim 1, wherein the composition further comprises at least one selected from the group consisting of an emollient, a surfactant, a preservative, and a pigment.

6. The solid cosmetic composition of claim 5, wherein the emollient is at least one selected from the group consisting of diisostearyl malate, dicetearyl dimer dilinoleate, hexyl laurate, squalane, pentaerythrityl tetraisostearate, octyldodecyl stearoyl stearate, dimethicone, and phenyl trimethicone.

7. The solid cosmetic composition of claim 5, wherein the emollient is contained in a content of 5 to 40 wt % relative to the total weight of the composition.

8. The solid cosmetic composition of claim 5, wherein the surfactant is at least one selected from the group consisting of glycerin fatty acid ether, propylene glycol fatty acid ester, sorbitan fatty acid ester, polyglyceryl-10 stearate, and cetearyl glucoside.

9. The solid cosmetic composition of claim 5, wherein the surfactant is contained in a content of 0.05 to 10 wt % relative to the total weight of the composition.
